(19) United States
(12) Patent Application Publication
(10) Pub. No.: US 2022/0080400 A1
(43) Pub. Date: Mar. 17, 2022

(54) HYDROFORMYLATION CATALYST, PREPARATION METHOD THEREFOR AND USE THEREOF

(71) Applicants: ZHEJIANG NHU COMPANY LTD., Shaoxing (CN); ZHEJIANG UNIVERSITY, Hangzhou (CN); SHANDONG NHU PHARMACEUTICAL CO., LTD., Weifang (CN); SHANDONG NHU VITAMIN CO., LTD., Weifang (CN)

(72) Inventors: Yong WANG, Hangzhou (CN); Zhe WANG, Hangzhou (CN); Yutong GONG, Hangzhou (CN); Lei WU, Shaoxing (CN); Lili YU, Shaoxing (CN); Haoran LI, Hangzhou (CN); Zhirong CHEN, Hangzhou (CN); Xiao MA, Weifang (CN); Jianyong MAO, Weifang (CN)

(21) Appl. No.: 17/258,439

(22) PCT Filed: Nov. 6, 2019
(86) PCT No.: PCT/CN2019/115850

§ 371 Date: Jan. 6, 2021

(30) Foreign Application Priority Data
Jun. 6, 2019 (CN) 201910492315.5

(51) Int. Cl. B01J 31/22; B01J 37/04

(52) U.S. Cl. CPC B01J 31/223; B01J 37/04; B01J 2531/822; B01J 2531/821; B01J 2231/321

(57) ABSTRACT

A hydroformylation catalyst, a preparation method thereof and a use thereof. The hydroformylation catalyst including an active component and a carrier carrying the active component, wherein the active component includes a transition metal as a center atom, and a polyhydroxy aromatic ring group bonded to the transition metal; and the transition metal and the polyhydroxy aromatic ring groups are bonded by at least one of metal-hydroxyl coordination bond and at least one of metal-oxygen covalent bond, and the active component has at least one of the metal-hydroxyl coordination bond and at least one of the metal-oxygen covalent bond.

### CROSS-REFERENCE TO RELATED APPLICATIONS

This application is a 371 of international PCT patent application PCT/CN2019/115850 filed on Nov. 6, 2019, which claims all benefits accruing under 35 U.S.C. § 119 China Patent Application No. 201910492315.5, tilled “HYDROFORMYLATION CATALYST, PREPARATION METHOD THEREFOR AND USE THEREOF”, filed on Jun. 6, 2019, in the China National Intellectual Property Administration, the content of which is hereby incorporated by reference.

### TECHNICAL FIELD

The present disclosure relates to catalyst, in particular to a hydroformylation catalyst, a preparation method thereof and a use thereof.

### BACKGROUND

A hydroformylation reaction of alkene is one of the main methods for preparing aldehydes. The hydroformylation reaction was first discovered by O. Roelen in 1938 at Ruhr Chemie Company in Germany. Cobalt carbonyl is as a catalyst for the first generation of hydroformylation reaction. During the reaction, Co₂(CO)₈ was first dissolved in a reaction liquid, the formed HCo(CO)₄ was regarded as the active species in the reaction. However, HCo(CO)₄ decomposed easily to produce CO, and only existed stably if under higher CO pressure.

In the second generation of hydroformylation reaction, phosphorus ligands were used for improving the stability of catalytically active species. It could effectively reduce the pressure required for the reaction and increase the proportion of normal aldehydes. However, phosphorus ligands would cause a decrease in catalyst activity and accelerate a hydrogenation reaction of alkenes and product aldehydes, resulting in a decrease in aldehyde selectivity.

In the third generation of hydroformylation reaction, an oil-soluble rhodium ligand complex catalyst was used as a catalyst. This type of catalyst could greatly improve efficiency of the hydroformylation reaction. For example, when a triphenylphosphine modified Rh-catalyst was used, conditions of reaction were mild, selectivity of a normal aldehyde was high, and side reactions such as alkene hydrogenation and the like were greatly reduced. However, because the product and the catalyst were in a homogeneous liquid phase, it was difficult to separate the product and the catalyst to recycle the catalyst; if distilling the product to separate the catalyst from the product, it would often lead to problems such as product polymerization, catalyst decomposition and deactivation.

In the fourth generation of hydroformylation reaction, a water-soluble phosphorus ligand and an oil-water two-phase reaction system were used. The catalyst was in the water phase and the product aldehyde was in the oil phase. After the reaction, the catalyst and the product could be effectively separated by standing. Because a low mass transfer efficiency of the two-phase reaction affected the catalytic effect, it was necessary to add a phase transfer agent. However, the addition of the phase transfer agent would cause emulsification and increase the difficulty of phase separation.

Thus, it is urgent to obtain a catalyst that has high activity and can be separated from the product and recycled.

### SUMMARY

A hydroformylation catalyst, a method for preparing the same, and use thereof should be provided.

The present disclosure provides a hydroformylation catalyst including an active component and a carrier carrying the active component. The active component includes a transition metal as a center atom, and a polyhydroxy aromatic ring group bonded to the transition metal. The transition metal and the polyhydroxy aromatic ring groups are bonded by at least one of a metal-hydroxyl coordination bond and a metal-oxygen covalent bond, and the active component includes at least one metal-hydroxyl coordination bond and at least one metal-oxygen covalent bond.

In one embodiment, the transition metal and one polyhydroxy aromatic ring group are bonded by both the metal-hydroxyl coordination bond and the metal-oxygen covalent bond, and the hydroxyl is preferably a phenolic hydroxyl.

In one embodiment, the active component has a structure as shown in Formula (1):

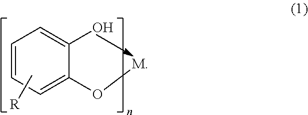

In Formula (1), M represents the transition metal, a bond between —OH and M is the metal-hydroxyl coordination bond, a bond between O atom and M is the metal-oxygen covalent bond, R is H atom or a substituent group, and n is larger than or equal to one and less than or equal to three. Preferably, the R is a substituent having a hydroxyl. When n is larger than one, the R on each benzene ring is independently selected from a group of H and the substituent group.

In one embodiment, n is three, and the active component has a structure of Formula (2):

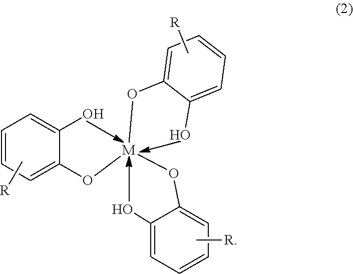

In one embodiment, the active component has a structure of Formula (3):

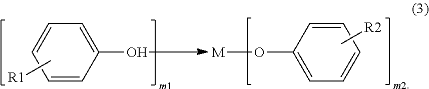

In Formula (3), M represents the transition metal, a bond between —OH and M is the metal-hydroxyl coordination bond, and a bond between O atom and M is the metal-oxygen covalent bond. R1 and R2 are independently selected from a group of H and substituent group. A sum of m1 and m2 is larger than or equal to one and is less than or equal to twelve, and m1 is larger than or equal to one, and m2 is larger than or equal to one. Preferably, the R1 and the R2 are independently selected from a group of substituents having a hydroxyl.

In one embodiment, the active components form a network with each other by the cross-linking of hydrogen bonds between the hydroxyls of the polyhydroxy aromatic ring groups.

In one embodiment, the active component and the carrier are bonded by a hydrogen bond formed by a hydroxyl of the polyhydroxy aromatic ring group of the active component and the carrier.

In one embodiment, a mass ratio of the active component and the carrier is in a range of 0.1:100 to 20:100.

The present disclosure further provides a method for preparing a hydroformylation catalyst including the following steps.

Providing a mixture including a carrier, a metal precursor, a polyhydroxy aromatic compound and a solvent, wherein the metal precursor dissociates in the solvent and forms a transition metal ion.

Adjusting a pH of the mixture to a range of 8 to 11 by an alkaline material to obtain the hydroformylation catalyst.

In one embodiment, the method for preparing the hydroformylation catalyst further includes a step of modifying the carrier with a chemical group, wherein the chemical group comprises at least one of hydroxyl group, sulfhydryl group and amino group.

In one embodiment, the carrier includes at least one carrier of activated carbon, silicon dioxide and metal oxide, and the metal oxide is at least one of Al₂O, MoO₃, WO₃, V₂O₅, VO₂, MgO and ZnO.

In one embodiment, the metal precursor comprises at least one of (NH₄)₂RuCl₆, RuCl₃, Cl₅H₂₁O₆Ru, H₁₂Cl₆N₃Rh, RhN₃O₉ and RhCl₃.3H₂O.

In one embodiment, the polyhydroxy aromatic compound includes two or more than two hydroxyls. The hydroxyl of the polyhydroxy aromatic compound is preferably a phenolic hydroxyl. The phenolic hydroxyl is preferably an ortho-phenolic hydroxyl.

In one embodiment, the polyhydroxy includes at least one of o-benzene-diol, m-benzene-triol and tannic acid.

In one embodiment, the mass ratio of the polyhydroxy aromatic ring group and the metal precursor is in a range of 0.1:1 to 50:1.

In one embodiment, the mass ratio of the carrier and the metal precursor is in a range of 5:1 to 1000:1.

The present disclosure further provides a use of a catalyst for a hydroformylation of in hydroformylation of alkene and alkyne.

In a hydroformylation catalyst of the present disclosure, the polyhydroxy aromatic ring group and the transition metal having catalytic activity are bonded to form the active component. The active component can be carried on a solid-phase carrier to heterogenize the homogeneous catalyst, so that the catalyst is easily separated from the product and recycled after the reaction, and can be directly used in gas-phase continuous production. In addition, due to the carrier, the steric hindrance around the transition metal is relatively large, the hydroformylation reaction has high selectivity, so that the distribution of linear chain product and branched chain product is effectively adjusted. The polyhydroxy aromatic ring group and the transition metal can be bonded by a metal-hydroxyl coordination bond, or a metal-oxygen covalent bond. So the formed active component has both metal-hydroxyl coordination bond and metal-oxygen covalent bond. Such bonding type is more stable than only coordinate bond, so that the transition metal as the central atom will not easily fall off, aggregate or run off. Furthermore, due to a synergistic effect of metal-oxygen covalent bond and metal-hydroxyl coordination bond, the transition metal shows unique electronic structure and geometric construction, further improving the activity of the catalyst.

In the method for preparing the hydroformylation catalyst of the present disclosure, the hydroformylation catalyst can be made from a metal precursor, which can dissociate and form a transition metal ion, a polyhydroxy aromatic compound and a carrier in a condition having a pH of 8 to 11. In a condition having a pH of 8 to 11, a part of the hydroxyl groups of the polyhydroxy aromatic compound dissociate and form the hydroxyl aromatic group, and the hydroxyl aromatic group of the hydroformylation catalyst can combine with the transition metal ions dissociated from transition metal. A part of the hydroxyls dissociates and forms negative oxygen ions, and further form non-coordinate covalent bonds with the transition metal ions, and the other part of the non-dissociated hydroxyls form coordinate bonds with the transition metal ions, so as to form the active components. The active components are carried by the carrier to obtain the hydroformylation catalyst. The method for preparing the hydroformylation catalyst in the present disclosure is simple and easy to operate. The produced hydroformylation catalyst is stable in structure and is easy to separate from the product and recycle.

### BRIEF DESCRIPTION OF THE DRAWINGS

FIG. 1 is a structural diagram of a hydroformylation catalyst in an embodiment of the present disclosure.

FIG. 2 is a transmission electron microscope photo of a hydroformylation catalyst in an embodiment of the present disclosure.

### DETAILED DESCRIPTION

In order to make the objectives, technical solutions and advantages of the present disclosure clearer, the present disclosure will be further described in detail hereinafter by embodiments and in conjunction with the accompanying drawings. It should be understood that the specific embodiments described herein are only used to explain the present disclosure, but not to limit the present disclosure.

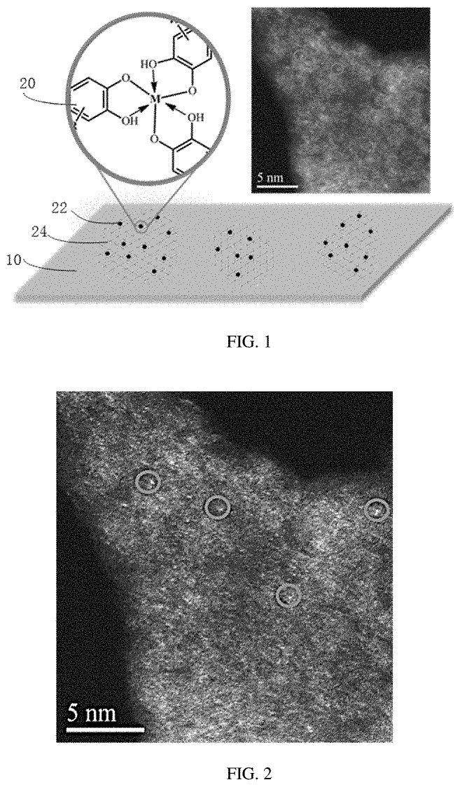

Referring to FIG. 1, the present disclosure provides a hydroformylation catalyst including an active component 20 and a carrier 10 carrying the active component. The active component 20 can include a transition metal 22 as a center atom, and a polyhydroxy aromatic ring group 24 bonded to the transition metal 22. The transition metal 22 and the polyhydroxy aromatic ring group 24 can be bonded by at least one of a metal-hydroxyl coordination bond and a metal-oxygen covalent bond, and the active component can include at least one metal-hydroxyl coordination bond and at least one metal-oxygen covalent bond.

In hydroformylation catalyst of the present disclosure, the polyhydroxy aromatic ring group and the transition metal having catalytic activity are bonded to form the active component. The active component can be carried on a solid-phase carrier to heterogenize the homogeneous catalyst, so that the catalyst is easily separated from the product and recycled after the reaction, and can be directly used in gas-phase continuous production. In addition, due to the carrier, the steric hindrance around the transition metal is relatively large, the hydroformylation reaction has high selectivity, so that the distribution of linear chain product and branched chain product is effectively adjusted. The polyhydroxy aromatic ring group and the transition metal can be bonded by a metal-hydroxyl coordination bond, or a metal-oxygen covalent bond. So the formed active component has both metal-hydroxyl coordination bond and metal-oxygen covalent bond. Such bonding type is more stable than only coordinate bond, so that the transition metal as the central atom will not easily fall off, aggregate or run off. Besides, due to a synergistic effect of metal-oxygen covalent bond and metal-hydroxyl coordination bond, the transition metal shows unique electronic structure and geometric construction, further improving the activity of the catalyst.

The polyhydroxy aromatic ring group can be preferably a rigid structure having a benzene ring, which makes a micro structure of the active component stable and not easy to deform.

The polyhydroxy aromatic ring group and the transition metal can be bonded by at least one of the metal-hydroxyl coordination bond and the metal-oxygen covalent bond, forming the active component having different structures.

In one embodiment, a transition metal and a polyhydroxy aromatic ring group can be bonded by both the metal-hydroxyl coordination bond and the metal-oxygen covalent bond, and the hydroxyl is preferably a phenolic hydroxyl. The active component can have a structure of Formula (1) as shown in below:

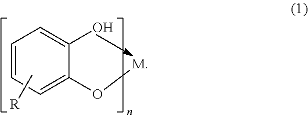

In Formula (1), M can represent the transition metal, a bond between —OH and the M can be the metal-hydroxyl coordination bond, a bond between O atom and the M can be the metal-oxygen covalent bond, and n can be larger than or equal to one and less than or equal to three. Preferably, n can be one or three, and more preferably, n can be three.

R can be H atom or a substituent group. The substituent group can be selected from a group of C1-C10 alkyl and C6-C20 aryl. Preferably, the R can be a halogen, amino, carboxyl or hydroxyl-substituted C1-C20 alkyl or a C6-C20 aryl. More preferably, the R can be a hydroxyl-substituted C1-C10 alkyl or C6-C20 aryl. The hydroxyl-substituted C1-C10 alkyl or C6-C20 aryl can have one or more hydroxyls, preferably has one to three hydroxyls.

When n is larger than one, the R on each benzene ring can be the same or not the same. The R on each benzene ring can be independently selected from a group of H and the substituent group.

Preferably, n can be three. The active component can be a structure of Formula (2) as shown in below:

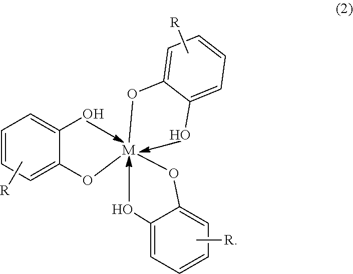

In another embodiment, an active component can be a structure of Formula (3) as shown in below:

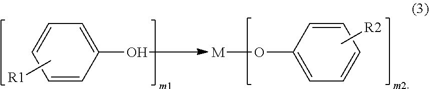

In Formula (3), M can represent for the transition metal. A bond between —OH and M can be the metal-hydroxyl coordination bond, and a bond between O atom and M can be the metal-oxygen covalent bond. A sum of m1 and m2 can be larger than or equal to one and less than or equal to twelve, and m1 can be larger than or equal to one, and m2 can be larger than or equal to one. Preferably, a sum of m1 and m2 can be larger than or equal to one or less than or equal to six.

The R1 and the R2 can be the same, or not. R1 and R2 can be independently selected from a group of the H and substituent group. The substituent group can be selected from a group of C1-C10 alkyls and C6-C20 aryls. Preferably, the R1 and R2 can be a halogen, amino, carboxyl or hydroxyl-substituted C1-C20 alkyl or a C6-C20 aryl. More preferably, the R1 and R2 can be a hydroxyl-substituted C1-C10 alkyl or C6-C20 aryl. The hydroxyl-substituted C1-C10 alkyl or C6-C20 aryl can have one or more hydroxyls, preferably one to three hydroxyls.

In one embodiment, the multiple active components can form a network by the cross-linking of the hydrogen bonds between the hydroxyls on the polyhydroxy aromatic ring groups. The transition metal is surrounded, so that it further prevents the transition metal from falling off, aggregating and missing and makes the catalyst more stable.

In one embodiment, the carrier can be modified with at least one of hydroxyl, sulfhydryl and amino on the surface thereof. The hydroxyl on the polyhydroxy aromatic ring groups of the active component and the carrier can be bonded by hydrogen bond. Due to the effect of the hydrogen bond, the active component and the carrier are bonded more firmly.

In one embodiment, a mass ratio of the active component and the carrier can be in a range of 0.1:100 to 20:100, preferably in a range of 0.2:100 to 2:100.

The present disclosure further provides a method for preparing a hydroformylation catalyst including the following steps.

S10, providing a mixture including a carrier, a metal precursor, a polyhydroxy aromatic compound and a solvent, wherein the metal precursor dissociates in the solvent and forms a transition metal ion.

S20, adjusting a pH of the mixture to a range of 8 to 11 by an alkaline material to obtain the hydroformylation catalyst.

In the method for preparing the hydroformylation catalyst of the present disclosure, the hydroformylation catalyst can be made from a metal precursor, which can dissociate and form a transition metal ion, a polyhydroxy aromatic compound and a carrier in a condition having a pH of 8 to 11. In a condition having a pH of 8 to 11, a part of the hydroxyl groups of the polyhydroxy aromatic compound dissociate and form the hydroxyl aromatic group, and the hydroxyl aromatic group of the hydroformylation catalyst can combine with the transition metal ions dissociated from transition metal. A part of the hydroxyls dissociates and forms negative oxygen ions, and further form non-coordinate covalent bonds with the transition metal ions, and the other part of the non-dissociated hydroxyls form coordinate bonds with the transition metal ions, so as to form the active components. The active components are carried by the carrier to obtain the hydroformylation catalyst. The method for preparing the hydroformylation catalyst in the present disclosure is simple and easy to operate. The produced hydroformylation catalyst is stable in structure and is easy to separate from the product and recycle.

The metal precursor can include at least one of (NH₄)₂RuCl₆, RuCl₃, C₁₅H₂₁O₆Ru, H₁₂C₁₆N₃Rh, RhN₃O₉ and RhCl₃.3H₂O. Preferably, the metal precursor can be RuCl₃ or RhCl₃.3H₂O.

The polyhydroxy aromatic compound can preferably have two or more than two hydroxyls, wherein the hydroxyl can be preferably a phenolic hydroxyl such as p-benzene-diol, o-benzene-diol, m-benzene-triol, tannic acid and the like, and more preferably, o-benzene-diol, tannic acid and the like. When the polyhydroxy aromatic compound can have an o-phenolic hydroxyl, the hydroformylation catalyst can have the active component having the structure of Formula (1). More preferably, the polyhydroxy aromatic compound can be tannic acid. Tannic acid having a large number of hydroxyls and hydroxyl bonds can be bonded more firmly to the carrier. Moreover, the polyhydroxy aromatic ring groups generated by the tannic acid are more easily cross-link with each other and form a network.

The carrier can be at least one of active carbon, silicon dioxide and metal oxide The metal oxide is an oxide of one or more elements selected from Al, Ti, Zr, Ce, Mo, W, V, Mg, Ca, Cr, Mn, Fe, Zn, Ga, Ge, Sn, Bi, Y, Nb, La and Re; preferably, from Al₂O₃, MoO₃, WO₃, V₂O₅, VO₂, MgO and ZnO. The shape of the carrier is not specifically limited. In one embodiment, the carrier can be particle-shaped. The particle-shaped carrier has a particle size in a range of 60 nm to 250 μm. In one embodiment, the catalyst can have a porous carrier, which has a porous size in a range of 0.2 nm to 100 nm.

In one embodiment, the method for preparing the hydroformylation catalyst can further include a step of modifying the carrier with chemical groups such as hydroxyl group, amino group, sulfhydryl group and the like. The surface of the carrier can be modified by chemical groups by chemical method or plasma processing. Due to the chemical groups on the surface of the carrier, the hydroformylation catalyst can be more firmly carried.

In one embodiment, the mass ratio of the polyhydroxy aromatic ring groups and the metal precursor can be in a range of 0.1:1 to 50:1. In one embodiment, wherein the mass ratio of the carrier and the metal precursor can be from 5:1 to 1000:1.

In the step of S10, the solvent can be able to dissolve the metal precursor and the polyhydroxy aromatic compound, and include water, ethanol, cyclohexane and the like.

When preparing the mixture, the mixture can be stirred or vibrated to mix evenly. In order to obtain a homogenous mixture, the metal precursor can be preferably firstly dissolved in the solvent to form a mixture, and then the carrier can be added into the mixture. Then the mixture can be mixed evenly by stirring or vibration, and then the polyhydroxy aromatic compound can be added and mixed.

In the step of S20, the alkaline material can be one or more selected from Na₂CO₃, NaHCO₃, NaOH and NH₃.H₂O. In order to prevent a precipitation generated, the solid alkaline material can be preferably added into the mixture in the form of a mixture having a certain concentration.

After pH of the mixture being adjusted to a range of 8 to 11, the mixture can be stood still at room temperature for 2 hours to 12 hours. Then the mixture can be filtrated, and an obtained solid can be washed and dried to obtain the hydroformylation catalyst. Preferably, the solid can be dried under a temperature in a range of 70 degree centigrade to 90 degree centigrade. Preferably, the drying time can be 24 hours to 48 hours.

The present disclosure further provides a use of the hydroformylation catalyst in hydroformylation of alkene and alkyne. For example, the hydroformylation catalyst can be used for hydroformylation of propene to produce n-butanol, 2-ethylheanol and other high alcohols; or can be configured for the hydroformylation of heptene to produce octanol; or can be configured for hydroformylation of a mixed alkene to produce C8-C10 alcohols and C12-C16 alcohols for plasticizer and synthetic detergent; and can be configured for hydroformylation of propyne to produce a monomer of the polymethyl methacrylate (MMA).

### Embodiment 1

20 mg RuC₃ was dissolved in 40 mL water, and 1 g active carbon was added, and the obtained mixture was stirred for 10 min. Then, 300 mg of tannin was added and stirred for 10 min.

NaOH mixture with a molar concentration of 0.1 mol/L was added in drop by drop to adjust the pH of the mixture to about 10, and continuously reacted for 2 hours.

A solid was obtained after the reaction. The solid was washed, and dried at 70 degree centigrade for 24 h to obtain a hydroformylation catalyst.

### Embodiment 2

Embodiment 2 was substantially the same as Embodiment 1, except that the metal precursor was RhCl₃.3H₂O.

### Embodiment 3

Embodiment 3 was substantially the same as Embodiment 1, except that the carrier of the catalyst was silicon dioxide.

### Embodiment 4

Embodiment 4 was substantially the same as Embodiment 1, except that the metal precursor was RhCl₃.3H₂O, and the carrier of the catalyst was silicon dioxide.

### Embodiment 5

Embodiment 5 was substantially the same as Embodiment 1, except that the polyhydroxy aromatic compound was o-benzene-diol.

### Embodiment 6

Embodiment 6 was substantially the same as Embodiment 1, except that the polyhydroxy aromatic compound was m-benzene-triol, and the carrier of the catalyst was silicon dioxide.

### Comparative Embodiment

A comparative embodiment was substantially the same as Embodiment 1, except that the pH of the mixture was in a range of 6 to 7.

The catalyst prepared by embodiments 1 to 6 can effectively catalyze the hydroformylation of alkene and alkyne. The catalytic effects of the catalysts for hydroformylation prepared in Embodiment 4 and Comparative Embodiment were shown herein after.

### Application Embodiment 1

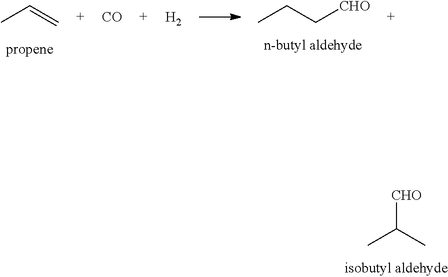

The catalysts for hydroformylation prepared in Embodiment 4 and Comparative Embodiment were used as the catalysts in formylation of propene, and the conditions of the reaction were the same. 50 mg catalyst, 10 mL deionized water, propene, carbon monoxide and hydrogen were added in a 500 mL autoclave, wherein a partial pressure of the propene was 0.4 MPa, a partial pressure of the carbon monoxide was 1 MPa, and a partial pressure of the hydrogen was 1 MPa. The reaction was taken at 100 degree centigrade for 6 hours. After the reaction, a test showed that when the catalyst was the catalyst obtained in Embodiment 4, the conversion ratio of the propene was 96%, selectivity of the n-butyl aldehyde aldehyde was 96%, and the molar ratio of the n-butyl aldehyde and the isobutyl aldehyde was 28:1. When the catalyst was the catalyst obtained in the Comparative Embodiment, the conversion ratio of the propene was 93%, selectivity of the n-butyl aldehyde aldehyde was 49%, and the molar ratio of the n-butyl aldehyde and the isobutyl aldehyde was 4:1, and the selectivity of propane was 39%.

### Application Embodiment 2

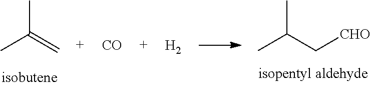

The catalysts for hydroformylation prepared in Embodiment 4 and Comparative Embodiment were used as the catalyst in formylation of isobutene, and the conditions of the reaction were the same. 50 mg catalyst, 10mL deionized water, isobutene, carbon monoxide and hydrogen were added in a 500 mL autoclave, wherein a partial pressure of the isobutene was 0.2 MPa, a partial pressure of a carbon monoxide was 1 MPa, and a partial pressure of the hydrogen was 1 MPa. The reaction was taken at 100 degree centigrade for 6 hours. After the reaction, a test showed that when the catalyst was the catalyst obtained in Embodiment 4, the conversion ratio of the isobutene was 98%, and selectivity of the isopentyl aldehyde was 97%. When the catalyst was the catalyst obtained in the Comparative Embodiment, the conversion ratio of the isobutene was 91%, selectivity of the isopentyl aldehyde was 54%, and the molar ratio of the isopentyl aldehyde and the neovaleraldehyde was 9:1, and the selectivity of isobutane was 40%.

### Application Embodiment 3

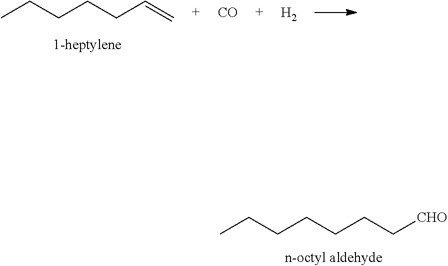

The catalysts for hydroformylation prepared in Embodiment 4 and Comparative Embodiment were used as the catalyst in formylation of 1-heptylene, and the conditions of the reaction were the same. 50 mg catalyst, 10 mL n-hexane, 30 mmol of 1-heptylene, carbon monoxide and hydrogen were added in a 500 mL autoclave, wherein the partial pressure of the carbon monoxide was 1 MPa, and the partial pressure of the hydrogen was 1 MPa. The reaction was taken at 100 degree centigrade for 8 hours. After the reaction, a test showed that when the catalyst was the catalyst obtained in Embodiment 4, the conversion ratio of the 1-heptylene was 99%, and selectivity of the n-octyl aldehyde was 98%. When the catalyst was the catalyst obtained in the Comparative Embodiment, the conversion ratio of the 1-heptylene was 82%, selectivity of the n-octyl aldehyde was 47%, and the molar ratio of the n-octyl aldehyde and 2-octyl aldehyde was 33:10, and the selectivity of n-heptane was 39%.

The technical features of the above-described embodiments may be combined in any combination. For the sake of brevity of description, all possible combinations of the technical features in the above embodiments are not described. However, as long as there is no contradiction between the combinations of these technical features, all should be considered as the scope of this disclosure.

The above-described embodiments are merely illustrative of several embodiments of the present disclosure, and the description thereof is relatively specific and detailed, but is not to be construed as limiting the scope of the disclosure. It should be noted that a number of variations and modifications may be made by those skilled in the art without departing from the spirit and scope of the disclosure. Therefore, the scope of the disclosure should be determined by the appended claims.

1. A hydroformylation catalyst comprising an active component and a carrier carrying the active component,
wherein the active component comprises a transition metal as a center atom, and a polyhydroxy aromatic ring group bonded to the transition metal; and
the transition metal and the polyhydroxy aromatic ring groups are bonded by at least one of a metal-hydroxyl coordination bond and a metal-oxygen covalent bond, and the active component has at least one metal-hydroxyl coordination bond and at least one metal-oxygen covalent bond.

2. The hydroformylation catalyst of claim 1, wherein the transition metal and the polyhydroxy aromatic ring group are bonded by both the metal-hydroxyl coordination bond and the metal-oxygen covalent bond.

3. The hydroformylation catalyst of claim 1, wherein the active component has a structure of Formula (1): 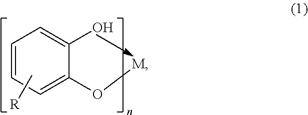
wherein, M represents the transition metal, a bond between —OH and M is the metal-hydroxyl coordination bond, a bond between O atom and M is the metal-oxygen covalent bond, R is H or a substituent group, and n is larger than or equal to one and less than or equal to three.

4. The hydroformylation catalyst of claim 3, wherein n is three, and the active component has a structure of Formula (2):

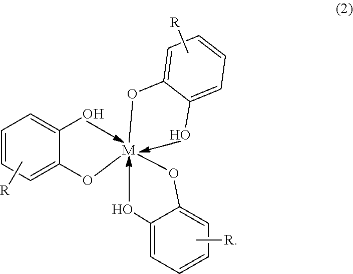

5. The hydroformylation catalyst of claim 1, wherein the active component has a structure of Formula (3): 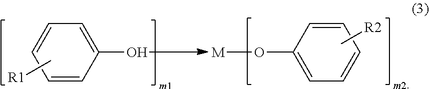
wherein in Formula (3), M represents the transition metal, a bond between —OH and M is the metal-hydroxyl coordination bond, a bond between O atom and the M is the metal-oxygen covalent bond, R1 and R2 are independently selected from a group of H and substituent group, a sum of m1 and m2 is larger than or equal to one and is less than or equal to twelve, m1 is larger than or equal to one, and m2 is larger than or equal to one.

6. The hydroformylation catalyst of claim 1, wherein the active components form a network with each other by cross-linking of hydrogen bonds between hydroxyls of the polyhydroxy aromatic ring groups.

7. The hydroformylation catalyst of claim 1, wherein the active component and the carrier are bonded by a hydrogen bond formed by a hydroxyl of the polyhydroxy aromatic ring group of the active component and the carrier.

8. The hydroformylation catalyst of claim 1, wherein a mass ratio of the active component and the carrier is in a range of 0.1:100 to 20:100.

9. A method for preparing a hydroformylation catalyst, the method comprising the following steps:
providing a mixture comprising a carrier, a metal precursor, a polyhydroxy aromatic compound and a solvent, wherein the metal precursor dissociates in the solvent and forms a transition metal ion; and
adjusting a pH of the mixture to a range of 8 to 11 by an alkaline material to obtain the hydroformylation catalyst.

10. The method of claim 9, further comprising a step of modifying the carrier with a chemical group, wherein the chemical group comprises at least one of a hydroxyl group, a sulfhydryl group and an amino group.

11. The method of claim 9, wherein the carrier comprises at least one of activated carbon, silicon dioxide and metal oxide, and the metal oxide comprises at least one of Al₂O₃, MoO₃, WO₃, V₂O₅, VO₂, MgO and ZnO.

12. The method of claim 9, wherein the metal precursor comprises at least one of (NH₄)₂RuCl₆, RuCl₃, C₁₅H₂₁O₆Ru, H₁₂C₁₆N₃Rh, RhN₃O₉ and RhCl₃.3H₂O.

13. The method of claim 9, wherein the polyhydroxy aromatic compound comprises two or more than two hydroxyls, and the hydroxyl of the polyhydroxy aromatic compound is a phenolic hydroxyl.

14. The method of claim 9, wherein the polyhydroxy aromatic compound comprises at least one of o-benzene-diol, m-benzene-triol and tannic acid.

15. The method of claim 9, wherein a mass ratio of the polyhydroxy aromatic compound and the metal precursor is in a range of 0.1:1 to 50:1.

16. The method of claim 9, wherein the mass ratio of the carrier and the metal precursor is in a range of 5:1 to 1000:1.

17. Use of the hydroformylation catalyst of claim 1 in hydroformylation of alkene and alkyne.

18. The hydroformylation catalyst of claim 1, and the hydroxyl is a phenolic hydroxyl.
